(19) United States
(12) Patent Application Publication
(10) Pub. No.: US 2024/0366804 A1
(43) Pub. Date: Nov. 7, 2024

(54) BIOCOMPATIBLE IMAGING PARTICLES, THEIR SYNTHESIS AND USE IN IMAGING TECHNIQUES

(71) Applicants: INSERM (INSTITUT NATIONAL DE LA SANTÉ ET DE LA RECHERCHE MÉDICALE), Paris (FR); UNIVERSITE DE CAEN NORMANDIE, Caen (FR); CENTRE HOSPITALIER UNIVERSITAIRE DE CAEN NORMANDIE, Caen (FR)

(72) Inventors: Thomas BONNARD, Caen (FR); Charlène JACQMARCQ, Caen (FR); Maxime GAUBERTI, Caen (FR); Sara MARTINEZ DE LIZARRONDO, Caen (FR); Denis VIVIEN, Caen (FR)

(21) Appl. No.: 18/291,666

(22) PCT Filed: Jul. 29, 2022
(86) PCT No.: PCT/EP2022/071455

§ 371 Date: Jan. 24, 2024

(30) Foreign Application Priority Data
Jul. 30, 2021 (EP) 21306071.8

(51) Int. Cl. A61K 49/18; A61K 49/08; A61K 49/16

(52) U.S. Cl. CPC A61K 49/1857; A61K 49/085; A61K 49/16

(57) ABSTRACT

The present invention relates to novel biocompatible imaging particles comprising superparamagnetic iron oxide (SPIO) assembled into submicromiter-sized clusters within a biodegradable polycathecolamine or polyserotonine matrix, their synthesis and use in imaging techniques. These particles overcome the issues of toxicity and unreliable signal of the molecules from the prior art by providing similar contrast to that of the microparticles of iron oxide and rapidly disassemble into isolated SPIO particles once they reach the acidic lysosomal compartment of the MPS cells, thus enabling their digestion. The present invention is thus directed to a particle having a hydrodynamic diameter comprised between 100 nm and 2000 nm, said particle comprising nanoparticles of iron oxide embedded within a matrix of polycathecolamine or polyserotonine, each of said nanoparticles of iron oxide being coated by a polymer which is different from polycathecolamine or polyserotonine

### INTRODUCTION

Despite significant progress in prevention and acute care over the past decades, ischemic stroke prevalence is on the rise with population aging and is expected to affect 1.3 million people per year in Europe by 2025.¹ While the rapid management of strokes saves the lives of half of the patients, the resulting brain damages often remains dramatic for survivors and ischemic stroke is the leading cause of acquired disability in adults.

The current treatment for the acute phase of ischemic stroke consists of eliminating the thrombus obstructing the cerebral circulation by injecting a drug promoting its enzymatic degradation (thrombolysis) or, since 2015,by removing it mechanically by catheterization (thrombectomy). However, even when successful recanalization of the main occluded vessel is achieved, downstream microcirculation often remain occluded.²

The mechanisms explaining this incomplete microvascular reperfusion are not fully understood but we know that it is due to an obstruction by microthrombi and that it is exacerbated by the inflammatory consequences of ischemia, which induces a narrowing of the lumen of the microvessels.³ Several preclinical and clinical studies correlate the presence of such microthrombi with cognitive declines and dementia.⁴ Recent retrospective analysis of the implementation of thrombectomy in ischemic stroke care also underlines the importance of incomplete microvascular reperfusion. More than a third of the patients who benefit from successful thrombectomy do not recover to functional independence although successful recanalization is rapidly achieved.⁵

Microthrombi in ischemic stroke are therefore of particular concern for patients surviving from ischemic stroke suffering permanent sequelae and thus represent a significant human, social and economic cost. Despite this concern, the impact of microvascular thrombosis in ischemic stroke is currently not properly considered in clinical practice. The main obstacle being the absence of reliable methods for microthrombi diagnosis within the brain of stroke victims. It is possible to evaluate their presence with microembolic signal in transcranial Doppler or identify the microlesions they induce in diffusion weighted MRI.⁶ Yet, this rely on the physiological disturbance eventually induced by the microthrombi rather than their actual detection and the diagnostic sensitivity is thus very poor.

A novel approach to reveal specifically and non-invasively the presence of microthrombi within the brain could significantly refine ischemic stroke diagnosis.

The technique of molecular imaging with microparticles of iron oxide (MPIO) have now been widely employed in preclinical settings to reveal vascular inflammation by magnetic resonance imaging (MRI).⁷⁻⁹ The MPIO accumulate at the area of the targeted disease epitope expression and reveal the pathology in T₂* weighted MRI thanks to their superparamagnetic properties. The technique has also been applied to image thrombosis in carotid artery¹⁰ and in lung embolism¹¹ but to date, none of these tool has shown the ability to reveal microvascular thrombosis within the brain.

Moreover, despite the great promises the strategy of molecular MRI represents for patient care, the toxicity of MPIO used in these studies preclude their use in humans. The one-micrometer diameter iron particles accumulate in the tissue from the mononuclear phagocytic system and do not degrade, causing lysosomal dysfunction and tissue vacuolation which represents a non-acceptable risk to induce liver dysfunction.¹²

On the other hand, similar superparamagnetic iron oxide (SPIO) particle presenting a smaller diameter (from 10 to 150 nm) are already approved for administration in human and used as a blood pool T₂* weight MRI contrast agent. Studies have shown that the SPIO injected into the blood stream are uptaken by the cells from the monocyte phagocytic system (MPS), digested in their lysosome and the iron content is finally metabolized by the organism.¹³

For this reason, many researchers tried to use those biocompatible SPIO for molecular imaging application but the contrast has always been too low to provide a reliable signal in T₂* weighted MRI. The nature of the susceptibility artefact used to detect superparamagnetic contrast agents (the blooming effect) requires a minimum concentration of iron within a voxel for reliable detection. Thus, a smaller diameter is required to allow metabolisation of the iron oxide but a larger diameter is needed to ensure reliable molecular imaging.

These issues of toxicity and unreliable signal have been successfully overcome by the inventors who developed a novel iron oxide platform comprising SPIO particles assembled into submicromiter-sized clusters within a biodegradable polycathecolamine or polyserotonine matrix. The clusters provide similar contrast to that of the MPIO and rapidly disassemble into isolated SPIO particles once they reach the acidic lysosomal compartment of the MPS cells, thus enabling their digestion.

### DESCRIPTION OF THE INVENTION

A first object of the invention is a particle having a hydrodynamic diameter comprised between 100 nm and 2000 nm, preferentially between 200 nm and 1500 nm, more preferentially between 300 nm and 1000 nm, even more preferentially between 500 nm and 1000 nm,

said particle comprising coated nanoparticles of iron oxide embedded within a matrix of polycathecolamine or polyserotonine,

each of said coated nanoparticles of iron oxide being coated by a polymer which is different from polycathecolamine or polyserotonine.

In an embodiment, the particle according to the invention has a hydrodynamic diameter comprised between 200 nm and 2000 nm, preferentially between 250 nm and 1500 nm, more preferentially between 300 nm and 1200 nm, even more preferentially between 300 nm and 1000 nm.

In an embodiment, the particle according to the invention has a hydrodynamic diameter comprised between 200 nm and 1000 nm, preferentially between 300 and 1000 nm, more preferentially between 500 and 1000 nm, and even more preferentially between 700 and 1000 nm.

Without wanting to be bound by any theory, the inventors believe that a hydrodynamic diameter within the range 200 to 2000 nm allow to obtain a signal. Besides, the inventors also consider that taking into account the risk of avoiding deleterious effects such as thrombotic effects for the patient in a context of visualization of thrombi, it is preferable to use particles having a hydrodynamic diameter of less than 2000 nm, and for safety reasons of less than 1000 nm. In view of the foregoing, the inventors believe that an optimal range of hydrodynamic diameter, allowing to have a satisfactory signal while avoiding the risk of deleterious effects in clinical situations would be comprised between about 700 nm and about 1000 nm.

As used herein, the expression “comprised between . . . and . . . ” should be understood to include the boundaries of the recited range.

In the context of the invention, the term “hydrodynamic diameter” refers to the diameter of a hypothetical hard sphere that diffuses with the same speed as the particle being measured. It reflects the size of the particle when in solution and includes coatings or surface modifications made to the particle in question.

The hydrodynamic diameter of the particles of the invention may be determined according to any method known by the person skilled in the art. In particular, it may be determined by dynamic light scattering (DLS), with for example a NanoZS® apparatus (Malvern Instruments, Worcestershire, UK) equipped with a 633 nm laser at a fixed scattering angle of 173°, with the temperature of the cell being kept constant at 25° C. The particles are for this measure put in suspension in water at a concentration of 20 μg to 200 μg of iron per mL of water.

Other know methods to determine the hydrodynamic diameter are particle tracking analysis (PTA) or its variant nanoparticle tracking analysis (NTA).

The particle of the invention allows combining the metabolization of iron oxide due to the small diameter of the nanoparticles, and reliable molecular imaging thanks to a larger diameter of the final particle which is an aggregate of nanoparticles within a biodegradable matrix of polycathecolamine or polyserotonine. Besides, the inventors have shown in vitro that when the particles of the invention, having a matrix of polydopamine, are not mixed with plasma, they are not able to recognize blood platelets. Without wanting to be bound by any theory, the inventors believe that when placed in the plasma, the matrix of polycathecolamine or polyserotonine, of the particles of the invention interacts with the plasma, and probably binds certain plasma proteins, resulting in the formation of a plasma protein corona around the particles of the invention. It is believed by the inventors that this plasma protein corona, which would form in situ in the plasma after injection of the particles of the invention, play a role in the targeting of the particles of the invention to the microthrombi.

Iron oxide nanoparticles incorporated into the particles of the invention can be chosen among maghemite of formula Fe₂O₃, magnetite of formula Fe₃O₄ or a mixture of Fe₂O₃ and Fe₃O₄. These different types of iron oxide are both superparamagnetic and biocompatible, allowing their use in particular as contrast agents in Magnetic Resonance Imaging (MRI) or as tracers in Magnetic Particle Imaging (MPI).

In the context of the invention, the term “biocompatible” refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

These nanoparticles are typically coated with a polymer which is different from polycathecolamine or polyserotonine. In particular, said coating polymer is chosen from a dextran, such as dextran, carboxydextran, or carboxymethyldextran, or a polyethylene glycol. It is to be noted that commercially available and FDA-approved coated iron oxide nanoparticles, as for example Resovist® from Bayer sold on the preclinical market by Magnetic Insight under the brand Vivotrax®, are available and well-suited to be incorporated into the particles of the invention. Other compatible commercially available coated iron oxide nanoparticles are easily available, such as SINEREM® or Endorem® from Guerbet, or nanomag®-D.

The diameter of coated iron oxide nanoparticles incorporated in the particles of the invention is preferentially chosen from 5 to 175 nm, more preferentially from 30 to 150 nm, even more preferentially from 50 to 75 nm.

The polymer matrix of the particle of the invention is selected from polycathecolamines or polyserotonine.

In particular, the biodegradable polycathecolamine matrix in the particles of the invention can be chosen among polydopamine (PDA), polynorepinephrine (PNE) or polyepinephrine (PEP), preferentially polydopamine. Polyserotonine (PST) can also be used as such biodegradable matrix.

In the context of the invention, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo. In some embodiments, biodegradable polymer materials break down into their component monomers.

In addition to their biodegradability, these different types of polymers have many advantages for the synthesis of the particles of the invention as well as their applications. They have the capacity to autopolymerize, which facilitates the synthesis. They form a strong bond to iron oxide which allows to obtain stable particles even under sonication, allowing dispersing aggregated particles without breaking the clusters or detaching conjugated ligands.

Indeed, a variety of ligand can be conjugated at high density with polycathecolamines or polyserotonine via Michael addition or Schiff base reactions (Lee, H. et al., Adv Mater. 2009, 21, 431-434). The ability to conjugate a large amount of targeting moieties on the surface of the particles of the invention can be advantageous in order to maximize the binding to a target and reach a higher sensitivity.

Polycathecolamines and polyserotonine are also hydrophilic and negatively charged at physiological pH, providing a negative zeta-potential for the coated particles and preventing their aggregation in solution.

These polymers also have antioxidant properties protecting iron oxide from oxidation reactions, which is advantageous because a better paramagnetic effect is obtained with magnetite compared to its oxidized form maghemite.

All these polymers have free amine groups enabling further functionalization, in particular with antibodies for a use in molecular imaging, but also other functional moieties such as polymer chains with terminal amination or various therapeutic molecule for drug delivery application. In the case of a functionalization of the particles of the invention with antibodies, a final coating, with glycine for example, can be carried out during the process of preparation in order to improve the solubility and stability of the final particles.

The nature of the polymeric matrix also enables to target the site to be visualized. In particular, in the case of microthrombi, the inventors were able to observe by bi-photon microscopy the mechanical retention of the particles of the invention on the edge of the microthrombi.

The particles of the invention can be characterized by their polydispersity index from 0.1 to 0.4, preferentially from 0.15 to 0.35.

The polydispersity index of the particle of the invention may be determined by any suitable known by the person skilled in the art. In particular, the polydispersity index of the particle of the invention may be determined by dynamic light scattering (DLS) by using for example the same apparatus and measurements conditions as those used for the measurement of the hydrodynamic diameter.

In the context of the invention, the term “polydispersity index” refers to a measure of the heterogeneity of a sample based on size. Polydispersity can occur due to size distribution in a sample or agglomeration or aggregation of the sample during isolation or analysis.

The particles of the invention can also be characterized by their zeta-potential ranging between −50 and −20 mV, preferentially between −45 and −25 mV.

The zeta-potential may be determined by any suitable known by the person skilled in the art. In particular, the zeta-potential of the particle of the invention may be determined by electrophoretic light scattering (ELS) with a measurement carried out on the particles suspended in a 1 mM sodium chloride solution.

In the context of the invention, the term “zeta-potential” refers to the electrical potential at the interface which separates mobile fluid from fluid that remains attached to the surface of a particle.

Another object of the invention is a suspension of particles according to the invention. Such a suspension of particles contains a solvent which can be selected from an aqueous solution, for example water, or saline solution, or glycerol, or mannitol in which the particles of the invention described above are suspended.

In the context of the invention, the term “suspension” refers to a heterogeneous mixture of materials comprising a liquid and a finely dispersed solid material.

The particles in the suspension of particles according to the invention have preferentially a mean hydrodynamic diameter comprised between 250 and 1000 nm, preferentially between 300 and 1000 nm, more preferentially between 500 and 900 nm, even more preferentially between 600 and 800 nm.

In an embodiment, the suspension of particles according to the invention have a mean hydrodynamic diameter comprised between 200 nm and 2000 nm, preferentially between 250 nm and 1500 nm, more preferentially between 300 nm and 1200 nm, even more preferentially between 300 nm and 1000 nm.

In an embodiment, the suspension of particles according to the invention have a mean hydrodynamic diameter comprised between 200 nm and 1000 nm, preferentially between 300 and 1000 nm, more preferentially between 500 and 1000 nm, and even more preferentially between 700 and 1000 nm.

Another object of the invention is the process of preparation of a suspension of particles of the invention comprising the steps of:
a) Mixing under agitation a solution of catecholamine or serotonine with coated iron oxide nanoparticles, thereby causing self-polymerization of catecholamine or serotonine and the formation of particles containing coated iron oxide nanoparticles embedded in a matrix of polymerized catecholamine or serotonine;
b) Terminating said polymerization;
c) Treating the resulting reaction mixture to obtain the final particles of the desired size;
d) Recovering a suspension of particles.

The polymerization step a) may be performed according to any suitable method known by the person skilled in the art. In particular, the step a) of the process of the invention can be carried out by mixing coated iron oxide nanoparticles suspension in an aqueous solution with a solution of a catecholamine, in particular dopamine, norepinephrine or epinephrine, or of serotonine. This step is carried out at a basic pH above 7, which can be ensured by the presence of any suitable basic solution, in particular a buffer, such as a TRIS buffer.

The constant agitation carried out during step a) allows to avoid a sedimentation of the reaction mixture. Such a sedimentation would thereby result in the formation of one big aggregate in the form of a paste which would be impossible to further treat.

The coated iron oxide nanoparticles suspension can typically have a concentration from 0.5 to 10 mg Fe/ml, particularly 1.5 mg Fe/ml. Coated iron oxide nanoparticles are typically in suspension in an aqueous solution, for example an aqueous solution of NaCl. Such aqueous solution of NaCl can typically be used at a concentration of 0.9% weight/volume, i.e. 9 mg of NaCl per ml of water.

The catecholamine or serotonine can typically be in solution in a buffer, such as a TRIS buffer, at a concentration from 5 to 100 mM, particularly 25 mM.

In step a), the coated iron oxide nanoparticles suspension and the solution of catecholamine or serotonine are typically mixed in a mass ratio Fe/(cathecolamine or serotonine) of 0.1 to 0.5, preferentially 0.2 to 0.4, more preferentially of about 0.3. For example, a mass ratio of 1.5 mg of iron for 4.8 mg of cathecolamine or serotonine can be used. When using a molar ratio, the coated iron oxide nanoparticles suspension and the solution of catecholamine or serotonine can typically mixed in a molar ratio Fe/(cathecolamine or serotonine) of 0.7 to 1.1, preferentially 0.8 to 1, more preferentially of about 0.9. For example, a molar ratio of 27 mmol of iron for 31 mmol of cathecolamine or serotonine can be used.
Step a) of the process of the invention comprises the formation of aggregates of coated iron oxide nanoparticles within the polycatecholamine or polyserotonine matrix. It can typically be carried out by stirring the reaction mixture of step a) at room temperature for 1 to 48 hours, preferentially 24 hours.
Step b) of termination of the polymerization reaction can typically be carried out by washing the obtained mixture with a solution at a pH from 8 to 10, approximately around 9, typically a buffer devoid of amine groups, for example a phosphate buffer.

This washing step consists in a replacement of the reaction medium. The solvent from step a) is removed by first separating the particles from the solvent, using a separating magnet or a centrifugation step, allowing to keep the particles in a bottom layer and the solvent as a supernatant. The solvent is then removed and replaced by the wash solution. This operation can be done multiple times to ensure that the solvent from step a) is totally removed and replaced by the wash solution.

This step leads to the termination of the polymerization reaction because monomers of catecholamine or serotonine are removed with the solvent during the washing step. Then, when there are no more monomers in the reaction mixture, the polymerization reaction ends.

Terminating step b) could be also be carried out by the addition of an acid to get an acidic pH in the reaction mixture, or also by the addition of a polymerization inhibitor.

The crude mixture obtained after step b) can be kept with no agitation at room temperature for a period of at least few hours prior to the final treatment of step c) in order to facilitate said future treatment step c).

In case where the treatment of step c) is carried out by sonication, the crude mixture obtained after step b) can be kept with no agitation at room temperature for a period of 8 to 3-hours, preferentially 12 to 30 hours, more preferentially of 24 hours;

The final treatment of step c) can be carried out for example by sonicating the resulting solution from step b) for 0.5 to 30 minutes, preferentially 15 minutes, to obtain the final particles of the desired size.

This step can be carried out with any classical sonicator, such as a sonicator probe, at high intensity, for example from 200 to 300 mV, preferentially at around 250 mV.

Treatment step c) is carried out on the reaction mixture resulting from step b), i.e. a suspension of the particles in the wash solution, typically a phosphate buffer.

At this stage, a separating magnet or a centrifugation step can be used again to keep the particles of the desired size in a bottom layer and the smaller ones can be discarded with the supernatant.

The final step d) consists in the recovery of a suspension of the particles of the invention. These particles can then be stored at a low temperature of about 3 to 10° C., either in the wash solution from step b) of the process or in water, or saline solution. The particles of the invention are preferentially stored with a constant slow agitation in order to limit the formation of aggregates.

Prior to their injection to a patient, the particles of the invention can be put under agitation, preferentially a vigorous agitation, in order to homogenize the suspension, at a temperature of 5 to 30° C., preferentially 8° C.

Another object of the invention is a suspension of particles or a particle obtainable by the process according to the invention.

Another object of the invention is a particle obtained by the process according to the invention after a additional step of isolation of said particle by removal of the solvent of the suspension obtained after step d).

The present invention also relates to a conjugate comprising a particle of the invention or a particle obtained by the process of the invention and a molecule comprising free amine or thiol groups, in particular a protein, a peptide, a nanobody, or a monoclonal antibody, or a molecule comprising a radiolabeled metal. In an embodiment, the conjugate comprises a particle of the invention or a particle obtained by the process of the invention and a molecule comprising at least a free amine or a thiol group. in particular a protein, a peptide, a nanobody, or a monoclonal antibody, a molecule comprising a radiolabeled metal, a small molecule such as N-acetyl cysteine. More particularly, the molecule comprising at least a free amine or a thiol group a protein may be a peptide, a nanobody, a monoclonal antibody, a molecule comprising a radiolabeled metal or N-acetyl cysteine. Even more particularly, the molecule comprising at least a free amine or a thiol group a protein may be a peptide, a nanobody or a monoclonal antibody. Preferably, the molecule comprising at least a free amine or a thiol group a protein may be a monoclonal antibody.

In an embodiment, the molecule comprising at least a free amine or a thiol group may be a molecule modified with a linker comprising at least a free amine or a thiol group. In another words, the molecule is a molecule wherein the free amine or thiol group is held by a linker moiety.

In the context invention, the term “linker” or linker moiety” means a connector allowing to link a molecule to a particle of the invention or a particle obtained by the process of the invention.

In the context of the invention, the term “conjugate” refers to a molecule composed of two or more molecules which are linked together. The conjugates of the invention are typically composed of a particle according to the invention linked to a protein, a peptide, a nanobody, or a monoclonal antibody. The particles of the invention can also be linked to radiolabeled metals, such as Copper⁶⁴, or Gallium⁶⁸. in particular in the context of a use in Positron Emission Tomography (PET) imaging technique.

The amount of particle according to the invention or obtained by the process of the invention in the conjugate may be comprised between 50% and 99%, in particular between 65% and 95%, more particularly between 80% and 90%, still more particularly between 80% and 90%, in moles with respect to the total molar amount of the conjugate.

Particularly, the monoclonal antibody can be chosen among immunoglobulin (Igg), vascular-cell adhesion molecule 1 (VCAM-1), intercellular adhesion molecule 1 (ICAM-1), P-Selectin, E-Selectin, or mucosal addressin cell adhesion molecule 1 (MAdCAM-1).

Particularly, the molecule comprising free amine or thiol group can be a fibrinolytic agent. In an embodiment, the fibrinolytic agent is bound to the particle of the invention, or a particle obtained by the process of the invention by a linker moiety having at least a free amine or thiol group.

Particularly, the molecule comprising free amine or thiol group is a protein that can be a tissue plasminogen activator or a fragment thereof, for example a recombinant tissue plasminogen activator (rtPA) such as alteplase, reteplase, or tenecteplase. In an embodiment, the protein that can be a tissue plasminogen activator or a fragment thereof is bound to the particle of the invention, or the particle obtained by the process of the invention by a linker moiety having at least a free amine or thiol group.

The conjugates can then be stored at a low temperature of about 3 to 10°0 C., either in the wash solution from step b) of the process or in water, or saline solution. The particles of the invention are preferentially stored with a constant slow agitation in order to limit the formation of aggregates.

Prior to their injection to a patient, the conjugates of the invention can be put under agitation, preferentially a vigorous agitation, in order to homogenize the suspension, at a temperature of 5 to 30° C. preferentially 8° C.

In order to be used in an in-vivo method of imaging, the particles of the invention or obtained by the process of the invention have to be administered, preferentially by injection, to a patient prior to the imaging step. This injection can be typically done intravenously, for example via a catheter in the arm vein (classically used for contrast agent administration), or also by intraarterial route.

Another object of the invention is therefore the particle or the conjugate of the invention or the suspension of particles of the invention or the particle or suspension obtained by the process of the invention for use in an in-vivo method of imaging.

The particles and conjugates of the invention and the particles obtained by the process of the invention have plural applications in the imaging field.

In particular, they can be used in imaging techniques such as Magnetic Resonance Imaging (MRI), Magnetic Particle Imaging (MPI), photoacoustic imaging, or Positron Emission Tomography (PET). They are particularly well-suited for Magnetic Resonance Imaging (MRI), Magnetic Particle Imaging (MPI), and photoacoustic imaging.

In the context of photoacoustic imaging, the absorption spectrum of the particles of the invention is particularly adapted to get a good visualization. Indeed, polydopamine for example absorbs mostly in the near infrared (wavelengths around 700 nm) and is well distinguished from oxygenated hemoglobin which absorbs mostly in the far infrared (wavelengths around 900 nm).

In the context of Positron Emission Tomography (PET), the particles of the invention or the particles obtained by the process of the invention can be used after a conjugation and/or a radiolabeling step. Radiolabeled metals can then be used, such as Copper⁶⁴, or Gallium⁶⁸.

All these techniques can be used for in-vivo diagnostic. The mode of action of the particles and conjugates of the invention and the particles obtained by the process of the invention make them suitable for all types of endovascular imaging. They can in particular be used in the diagnostic of microthrombi without a prior coupling to any functional moieties or in the diagnostic of vascular inflammation, for example in the brain, heart, lungs, kidneys and intestinal mucosa, with a prior coupling to antibodies targeting specifically biomarker of vascular inflammation (such as VCAM-1, P-Selectin, MAdCAM-1).

The experiments carried out by the inventors, which are further detailed in the Examples, demonstrate that due to their specific retention in microthrombi, the particles and conjugates of the invention and the particles obtained by the process of the invention are efficient to target microemboli within the microcirculation with no need of specific targeting moiety.

Experiments also demonstrated that the particles and conjugates of the invention and the particles obtained by the process of the invention are efficient to monitor thrombolysis: they are able of both identifying situation for which thrombolysis should be administered and subsequently verifying the efficacy of thrombolysis therapy.

The particles and conjugates of the invention and the particles obtained by the process of the invention have been shown to be very useful, in the context of a condition called disseminated intravascular coagulation (DIC), in revealing on MRI scans the microthrombi present in the whole body. DIC is a condition characterized by thrombosis in small blood vessels that usually develops in response to another disorder (such as cancer, sepsis, infectious disease) or event (such as organ transplant, trauma) which disrupt the coagulation system.²¹ It is for instance one of the severe complications identified in patients with pneumonia implied in infectious diseases such as the COVID-19.²² The current diagnosis method for DIC is limited to the detection of coagulation dysregulation in the blood.²³

The particles and conjugates of the invention and the particles obtained by the process of the invention are also efficient to reveal microthrombi formed in a context of ischemia-reperfusion. The abrupt removal of the filament obstructing the middle cerebral artery for an ischemia of 60 minutes triggers the coagulation system. This situation of abrupt reperfusion is often encountered for ischemic stroke patients undergoing endovascular thrombectomy (EVT).²⁴

The present invention is also directed to an in-vivo diagnostic method using the particles or conjugates of the invention or the suspension of particles of the invention, or the particles or suspension obtained by the process of the invention.

Another object of the invention is a method of imaging wherein a patient has been administered, for example by injection, a composition containing the particles or conjugates of the invention or the suspension of particles of the invention, or the particles or suspension obtained by the process of the invention and comprising an imaging step.

In the context of the invention, the term “patient” refers to a warm-blooded animal, more preferably a human, who/which is awaiting or receiving medical care or is or will be the object of a medical procedure.

The term “human” here refers to subjects of both genders and at any stage of development (i.e. neonate, infant, juvenile, adolescent, adult). In one embodiment, the human is an adolescent or adult, preferably an adult.

In the context of the invention, the term “administration”, or a variant thereof (e.g., “administering”) refers to the provision of an active agent or active ingredient, alone or as part of a pharmaceutically acceptable composition, to the patient in whom/which the condition, symptom, or disease is to be treated, attenuated, visualised or diagnosed.

Another object of the invention is a composition containing the particles or conjugates of the invention or the suspension of particles of the invention or the particles or suspension obtained by the process of the invention. This composition can optionally contain at least one pharmaceutically acceptable excipient, carrier, diluent, and/or adjuvant.

Such a composition can contain a suspension of the particles or conjugates of the invention or the particles obtained by the process of the invention in a solvent, such as for example a solution of mannitol 0.3 M.

Prior to their injection, the particles of the invention can be suspended in any physiological medium compatible with an injection to a human patient. Examples of such physiological medium are mannitol or glycerol.

In the context of the invention, the terms “pharmaceutically acceptable” refer to ingredients of a pharmaceutical composition which are compatible with each other and not deleterious to the patient thereof.

The term “excipient” as used herein means a substance formulated alongside the active agent or active ingredient in a pharmaceutical composition or medicament. Acceptable excipients for therapeutic use are well known in the pharmaceutical art. The choice of excipient can be selected with regard to the intended route of administration and standard pharmaceutical practice. The excipient must be acceptable in the sense of being not deleterious to the recipient thereof. The at least one pharmaceutically acceptable excipient may be for example, a binder, a diluent, a carrier, a lubricant, a disintegrator, a wetting agent, a dispersing agent, a suspending agent, and the like.

Another object of the invention is the use of the particles or conjugates of the invention or the suspension of particles of the invention or of the particles or suspension obtained by the process of the invention as a contrast agent or as a tracer in an imaging method.

### FIGURES

FIG. 1A-F. Scheme and pictures of microparticles of lum injected intravenously which accumulate specifically at the area of occlusive microthrombi.

A. Scheme of the experimental design. Ischemic stroke was induced via injection of thrombin (1 μL, 1 U/μL) into the middle cerebral artery. Biphoton microscopy was performed over the downstream microcirculation. Brain microvessels were visible in the 647 nm channel shown in white on the images (B, C and F). Leukocytes and platelets were labelled with intravenous injection of rhodamine 6G (1 mg/mL), revealing the presence of microthrombi within the arterioles on the 558 nm channel presented in red on the images (B, D and F). FITC fluorescent microparticles (réf) were injected intravenousl and their accumulation at the microthrombi area was observed in the 488 nm channel shown in green on the images (E and F). Scale bars B: 100 μm, C, D, E and F: 20 μm.

FIG. 2. Schematic representation of a particle of the invention with a polydopamine matrix, and a simplified representation of its process of preparation.

FIG. 3A-J. Schemes and pictures of molecular MRI of microthrombi using the particles of the invention in a thrombin induced thromboembolic ischemic stroke model.

A. Coronal, sagittal and transverse sections from a 3D T2* weighted acquisition performed 15 minutes after ischemic stroke induction. B. Corresponding coronal, sagittal and transverse sections from the same 3D T2* weighted acquisition performed 1 minute post intravenous injection of PHysIOMIC contrast agent (1.5 mg Fe/kg), 25 minutes after ischemic stroke induction. C. Signal void quantification in the ipsi lateral brain area before versus after the injection of the PHysIOMICs (n=5). D. 3D reconstruction of the PHysIOMIC signal uptake shown in green color. E. Kinetic of the signal uptake before injection versus 1 h, 6 h, 12 h, 18 h and 24 h post PHYSIOMIC injection. F. Signal void quantification in the ipsi lateral brain area against time post PHysIOMIC injection. Lesion sized was measured at 24 h post stroke on T2-weighted sequences after the injection of saline (G) versus PHysIOMIC (H). I. Mean lesion sized at 24 h of animals injected with saline versus PHysIOMIC. J. The localisation of the PHysIOMIC microparticles were studied on histological sections of the brain at 1 h post stroke. PERLS staining reveal in blue color the presence of iron and confirmed the presence of the PHysIOMIC iron oxide microparticles around the microthrombi.

FIG. 4A-G. Schemes and pictures of thrombolysis of microthrombi monitored by molecular MRI using the particles of the invention.

A. Scheme of the thrombolysis protocol. Ischemic strokes were induced via injection of thrombin (1 μL, 1 U/uL) in the MCA. 10 minutes after, PHysIOMIC contrast agent was injected intravenously (1.5 mg Fe/kg). At 12 minutes post stroke induction, PHysIOMIC particles are injected and a first 3D T2* weighted MRI acquisition was performed to identified the microthrombi formed. Thrombolysis was then initiated 20 min after stroke induction via intravenous injection of tissue-type plasminogen activatore (tPA, Actilyse, 10 mg/kg) versus saline control (n=4). A total of 200 μL was injected, 20 μL injected as initial bolus and 180 μL at a slow perfusion rate. At 1 h post stroke induction, a second 3D T2* weighted MRI acquisition was performed to measure the amount of microthrombi remaining. At 24 h post stroke, a T2 weighted MRI acquisition was performed to measure the size of the brain lesion. Consecutive corronal sections from the 3D T2* weighted MRI acquisitions are presented, before and after thrombolysis with saline (B) or with tPA (C). Signal void quantification in the ipsi lateral brain area (n=4). E. T2 weighted MRI acquisition showing brain lesion at 24 h after thrombolysis treatment with saline or tPA. G. Mean lesion size measured at 24 h.

FIG. 5. Schemes and pictures of molecular MRI of brain microvascular thrombosis in other models using the particles of the invention.

A. 2 μg of staurosporin was injected in the right striatum via a steretoaxic injection with a glass micropipette, inducing local apoptosis that result in the formation of thrombosis. Coronal sections from a 3D T2* weighted acquisitions performed before and after the injection of PHysIOMIC microparticles. The signal void quantification confirmed signal uptake in the right striatum area. B. A monofilament was inserted through the exterbal carotid artery (ECA) and gently advanced to occlude the middle cerebral artery (MCA) at the bifurcation. The surgical wound was closed and the filament was left in place for 60 min. The filament was then removed to restore blood flow. Coronal sections from a 3D T2* weighted acquisitions performed before and after the injection of PHysIOMIC microparticles revealed the presence of microvascular thrombosis.

FIG. 6A-C. A. Transmission electronic microscopy observation of SPIO and PHysIOMIC suspensions. B. Dynamic Light Scattering analysis of SPIO and PHySIOMIC suspensions showing hydrodynamic size distribution measured by intensity. C. Mean values of the mean hydrodynamic size, polydispersity index and zeta potential are presented (n=3).

FIG. 7A-E. PHysIOMIC-induced hyposignal decreases in thrombolysed mice. A. Scheme of the thrombolysis protocol. Ischemic strokes were induced via injection of thrombin (1 μL. 1 U/μL) in the MCA. 10 minutes after, PHysIOMIC contrast agent was injected intravenously (1.5 mg Fe/kg). At 20 minutes post stroke induction, a first 3D T2* weighted MRI acquisition was performed to identified the microthrombi formed. Thrombolysis was then initiated 30 min after stroke induction via intravenous injection of tissue-type plasminogen activatore (tPA, Actilyse, 10 mg/kg) versus saline control (n=4). A total of 200 μL was injected, 20 μL injected as initial bolus and 180 μL at a slow perfusion rate. At 70 min post stroke induction, a second 3D T2* weighted MRI acquisition was performed to measure the amount of microthrombi remaining. At 24 h post stroke, a T2 weighted MRI acquisition was performed to measure the size of the brain lesion. B. PHysIOMIC were injected 10 min after occlusion and T2*-weighted sequence were acquired before and after treatment either with tissue-type plasminogen (tPA, 10 mg/kg) or saline. C. 3D reconstruction of a tPA treated mouse before and after the treatment with tPA. D. Quantification of induced hyposignal reveals a diminution in the treated group (n=8). E. Representative Magnetic Resonance Angiography obtained from time of flight weighted MRI sequences and mean angiographic score (n=8).

FIG. 8A-C. A. PHysIOMIC microparticles show a biodistribution and a biodegradation in the liver and spleen in full body MRI.

B. T2-weighted images were acquired before and after injection of PHySIOMIC and USPIO at 4 mg/kg, and longitudinaly at 2, 7 and 31 days, hyposignal in the liver and spleen decreases. B. Quantification of T2-values in the liver and spleen.

C. Transmission electronic microscopy (TEM) images of liver section after injection and at 2, 7 and 31 days after injection of PHysIOMIC at 4 mg/kg.

### EXAMPLES

The following study describes the synthesis of a contrast agent according to the invention obtained from the self-assembly of a FDA approved SPIO (resovist®, Bayer) and reports a method to reveal brain microvascular thrombosis on T₂* weighted MRI sequences thanks to the intravenous injection of the contrast agent. The imaging capacities of this diagnostic tool have been studied on 3 mouse models characterized by the presence of microvascular thrombosis in the brain, induced via different pathways. Microthrombi were examined by bi-photon microscopy and the mechanical retention of particles on the edge of the microthrombi was observed. Finally, this study demonstrates that the contrast agent according to the invention could be used to monitor thrombolysis therapy with tissue-type plasminogen, efficient to lyse the microthrombi.

In the following part, examples of particles according to the invention are denominated by the term “PhySIOMICs”.

### Example 1

### Material and Method

### Preparation of PHysIOMICs

### Synthesis and Analysis of Particles

PhySIOMICs are aggregates of biocompatible and superparamagnetic iron oxide (SPIO) nanoparticles (in this example VivoTrax™, Magnetic Insight, Inc., Alameda, CA), similar to SPIO nanoparticles approved for clinical imaging to detect liver carcinoma16, organized in a polydopamine structure. Briefly, SPIO nanoparticles suspension in an aqueous 0.9% solution of NaCl (1.5 mg Fe/mL) is mixed with a solution of cathecol amine (25 mM, dopamine, serotonine or norepinephrine)) in a TRIS buffer 10 mM pH 8.8.

Dopamine solution was prepared from dopamine hydrochloride (Sigma-Aldrich) at 10 mg/mL in water and added to a final concentration of 4.8 mg/mL in TRIS buffer 4.8 mM pH 8.8.

Serotonine solution was prepared from serotonine hydrochloride at 20 mg/mL in water and added to a final concentration of 5.3 mg/mL in TRIS buffer 4.8 mM pH 8.8 supplemented with ammonia (1.3% v/v).

Norepinephrine solution was prepared from norepinephrine bitartrate at 40 mg/mL in water and added to a final concentration of 8 mg/mL in TRIS buffer 10 mM pH 8.8 supplemented with ammonia (2.6% v/v).

Polymerisation of the cathecol amine into polydopamine (PDA), polyserotonine (PST) or polynorepinephrine (PNE) occurs under an Ultra-Turrax agitation (9500 rpm; IKA Instruments) for 2 hours and the reaction is continued under constant agitation at room temperature for 24 h. To stop polymerization, aggregates of nanoparticles are washed in PB 10 mM pH 8.8 using a separating magnet (PureProteome™ Magnetic Stand, Millipore). The solution is then placed under sonication at high intensity during 15 min to obtain particles of wanted size. PHysIOMICs are kept under agitation at −4° C. until injection.

A schematic representation of the particles of the invention is represented in FIG. 2 for a polydopamine PHysIOMIC particle.

PHysIOMICs were observed by confocal microscopy (Leica, SP5). Polymers of cathecol amine are materials with light reflexion properties. The 3 types of PHysIOMICs were observed from the reflexion of a 488 nm laser in the 488 nm channel.

Dynamic light scattering (DLS) was used to determine the average hydrodynamic diameter, the polydispersity index (PDI) and the diameter distribution by volume of PHysIOMICs suspensions using a NanoZS® apparatus (Malvern Instruments, Worcestershire, UK) equipped with a 633 nm laser at a fixed scattering angle of 173°. The temperature of the cell was kept constant at 25° C. and all dilutions were performed in pure water. Measurements were performed in triplicate.

Total iron was quantified using a modified version of the ferrozine colorimetric assay. 500 μL of 2N HCL was added to 500 μL of sample lysate. The iron standards were prepared using analytical grade of FeCl₂. Samples were then incubated overnight. Samples were then mixed with iron detection reagent (37.5 μL of 5 mM ferrozine, 60 μL of ammonium acetate 30% and 30 μL of ascorbic acid 30%; Sigma-Aldrich,). Equal volumes of the test and standard samples were aliquoted into a 96-well microplate in duplicate and absorbance was read at 560 nm using a microplate reader (ELx808 Absorbance Reader, Biotek Instruments).

### Mice

All studies were conducted on male Swiss mice (age 8-10 weeks; weight 35-45 g; CURB, Caen, France) in accordance with European communities Council (Directives of Nov. 24, 1986 (86/609/EEC) and French Legislation (act no, 87-848) on Animal Experimentation and validated by the local ethical comittee of Normandy (CENOMEXA). Mice were housed in a temperature-controlled room on a 12-hour light/12-hour dark cycle with food and water ad libitum. During surgery, mice were deeply anesthetized with isoflurane 5% in a 70%/30% gas mixture (NO2/O2) and maintained under anesthesia with 2% isoflurane in a 50%/50% gas mixture (NO2/O2). Rectal temperature was maintained at 37±0.5° C. throughout the surgical procedures using a feedback-regulated heating system. A catheter was inserted into the tail vein of mice for intravenous administration of PHysIOMICs. After surgery, animals were allowed to recover in a clean heated cage.

### Mouse Models

### Middle Cerebral Artery Occlusion (MCAO) With Thrombin or AlCl3

As described in Orset et al¹⁷, mice were placed in a stereotaxic device and the skin between the right eye and the right ear was incised, and the temporal muscle was retracted. A small craniotomy was performed, the dura was excised, and the middle cerebral artery (MCA) was exposed. A custumer-made glass micropipette was introduced into the lumen of the MCA and 1 μL of purified murine alpha-thrombin (1 UE; Stago BNL) was pneumatically injected to induce the in situ formation of the clot. The pipette was removed 10 minutes after the injection at which time the clot was stabilized. For the AlCl₃ MCAO, the MCA was exposed and AlCl3 (Sigma-Aldrich) was topically applied on the artery (as previously described¹⁸). Except during thrombolysis procedures, cerebral blood velocity was determined by laser Doppler flowmetry using a fiberoptic probe (Oxford Optronix). In order to expose the animals to the same concentration of gaseous anesthesia, all animals were kept under anesthesia for 1 hour after MCAO. For the study of thrombolysis effects on microthrombosis, mice received intravenous administration of tPA (10 mg/kg in 200 μL; Actilyse) as 10% bolus and 90% perfusion over 40 minutes after injection of alpha-thrombin. The control group received the same volume of saline under the same conditions.

### Transient Middle Cerebral Artery Occlusion With Intraluminal Filament

The intraluminal filament transient middle cerebral artery occlusion (tMCAO) model was performed on rats following a previously described protocol.¹⁹ Mice were placed in a supine position and a midline incision was performed in the neck. The right carotid bifurcation was exposed and the external carotid artery (ECA) was coagulated. A 6-0 monofilament (diameter 0.09-0.11 mm, length 20 mm; Doccol, MA, USA) was inserted through the ECA and gently advanced to occlude the MCA at the bifurcation. The surgical wound was closed and the filament was left in place for 60 min. The filament was then removed to restore blood flow and the internal carotid artery was ligated.

### Stereotaxic Injection of Staurosporine

A unilateral striatal injection of staurosporine (2 μg in a volume of 1 μL; Alfa Aesar™), a protein kinase inhibitor, was performed after placing mice in a stereotaxic frame at the following coordinates: 0.5 mm anterior, 2.0 mm lateral, 3.0 mm ventral to the bregma. The staurosporine solution was injected by the use of a glass micropipette (calibrated at 15 mm/μL).

### Magnetic Resonance Imaging (MRI)

All experiments were carried out on a Pharmascan 7 T/12 cm system with surface coils (Bruker, Germany). 3D T2*-weighted gradient echo imaging with flow compensation (GEFC, spatial resolution of 93×70×70 μm interpolated to an isotropic resolution of 70 μm) with TE/TR 9/50 ms and a flip angle of 15° was performed before and after the injections of PhySIOMIC contrast agent. PHysIOMIC suspensions were prepared to a concentration of (1.5 mg Fe/mL) and slowly injected as a single bolus intravenously via a tail vein catheter at 1.5 mg/kg. Brain lesion was measured on T₂-weighted images acquired using a multi-slice multi-echo (MSME) sequence (TE/TR 50/3000 ms with 70×70×500 μm3 spatial resolution.

### Image Analysis

Analysis of the MCA MRA were performed blinded to the experimental data using the score: 2: normal appearance, 1: partial occlusion, 0: complete occlusion of the MCA. Lesion sizes were quantified on T₂ weighted images using ImageJ software (v1.45r). All T₂*-weighted images presented in this study are minimum intensity projections of? consecutive slices (yielding a Z resolution of? μm). Signal void quantification on 3D T2*-weighted images, and 3D representation of PhySIOMICs-induced hyposignal were realised using automatic Otsu thresholding in ImageJ software. Results are presented as volume of MPIO-induced signal void divided by the volume of the structure of interest (in percent). Perfusion index (ΔR2* peak ratio) was calculated by measurement of the ratio of ipsilateral and contralateral ΔR2* as described previously²⁰, using a in-house-created macro, also in ImageJ.

### In Vivo Two-Photon Microscopy

Anesthetized mice used for two-photon experiments underwent thin-skull cranial window for the cortical in vivo detection of leukocyte rolling and adhesion. The head skin was opened to expose the skull and the right parietal bone was completely polished with a drill to leave only a thin layer of bone enabling the visualization of cortical cerebral blood vessels by transparency. Anesthetized mice were placed in a stereotaxic device and aqueous medium was deposed between the thin-skull window and the X25 immersive objective. One hundred μl of Rhodamine 6G (1 mg/kg. Sigma Aldrich) and 100 μl of NH₂-Cy₅ (5 mg/ml, Lumiprobe) were injected in the tail vein to stain circulating leukocytes and to visualize the lumen of blood vessels, respectively. Acquisitions were performed using a Leica TCS SP5 MP microscope at 840 nm two-photon excitation wavelength (Coherent Chameleon, USA). Photomultiplier (PMT) 2 (recorded capacity: 500-550 nm; gain 850V; offset 0) and PMT3 (recorded capacity: 565-605 nm; gain 850V; offset 0) were used. The pulsing laser characteristics were: gain 23%; trans 17%; offset 50%. FITC-marked microparticles (FITC=Fluorescein isothiocyanate) based on melamine resin (100 μL. Sigma Aldrich) were injected intravenously and their interaction at microthrombi area was observed.

### Immunohistochemistry and Histological Staining

Deeply anesthetized mice were perfused transcardially with saline followed by a fixative solution (4% paraformaldehyde in phosphate buffer) at a physiological rate (8 mL/min) with a peristaltic pump. Brains and liver were post-fixed (24 h, 4° C.) and cryoprotected (20% sucrose, 24 h, 4° C.) before freezing in Tissue-Tek (Miles Scientific). Coronal brain sections (10 μm) were stained with Perls' Prussian Blue and nucleus red (Leica Biosystems, Iron Kit stains) to detect and identify ferric (Fe³⁺) iron residue of PhySIOMICs particles. Images were digitally captured using a Leica DM6000 microscope-coopled coolsnap camera, visualized with Meta Vue 5.0 software and were further processed using QuPath and ImageJ. All analyses were performed blinded to the experimental groups.

### Statistical Analysis

All results are presented as mean ±SEM. Statistical analysis were performed using GraphPad Prism V8 (GraphPad software). Differences were considered statistically significant if probability value p<0.05.

### RESULTS

### Specific Retention of the Particles in Microthrombi

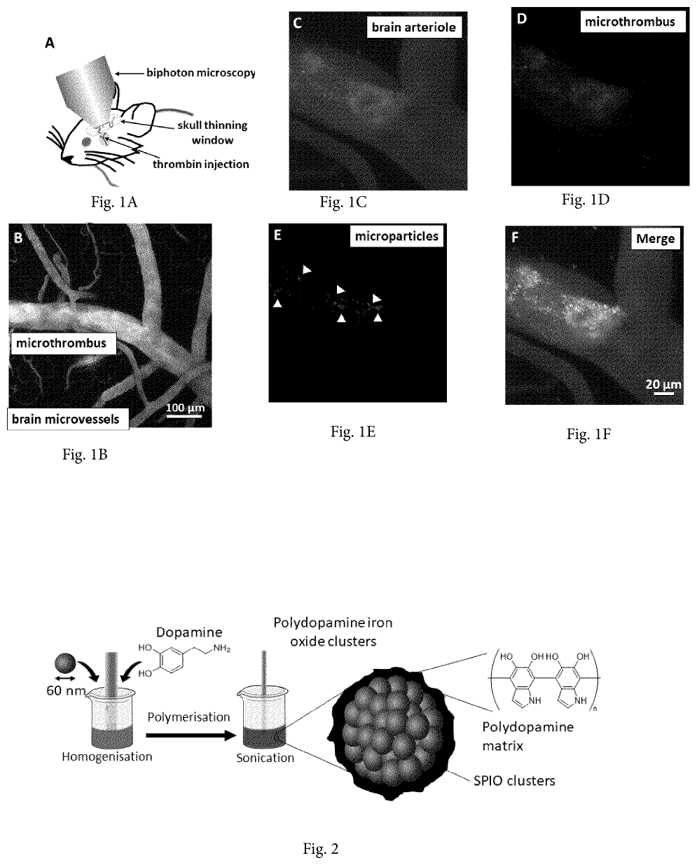

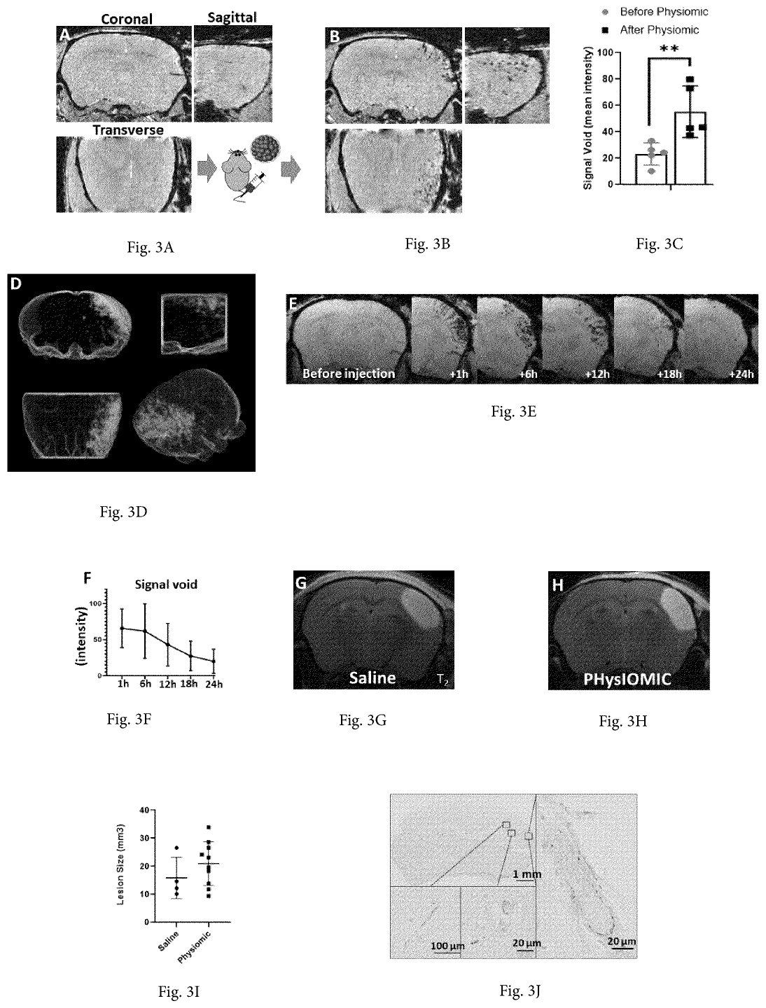

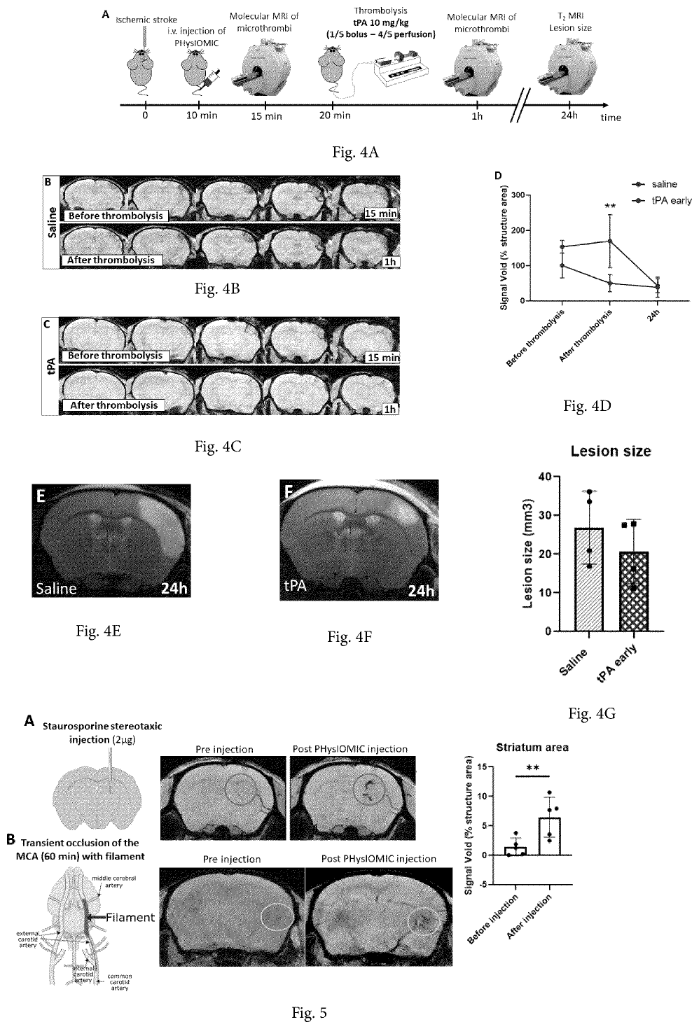

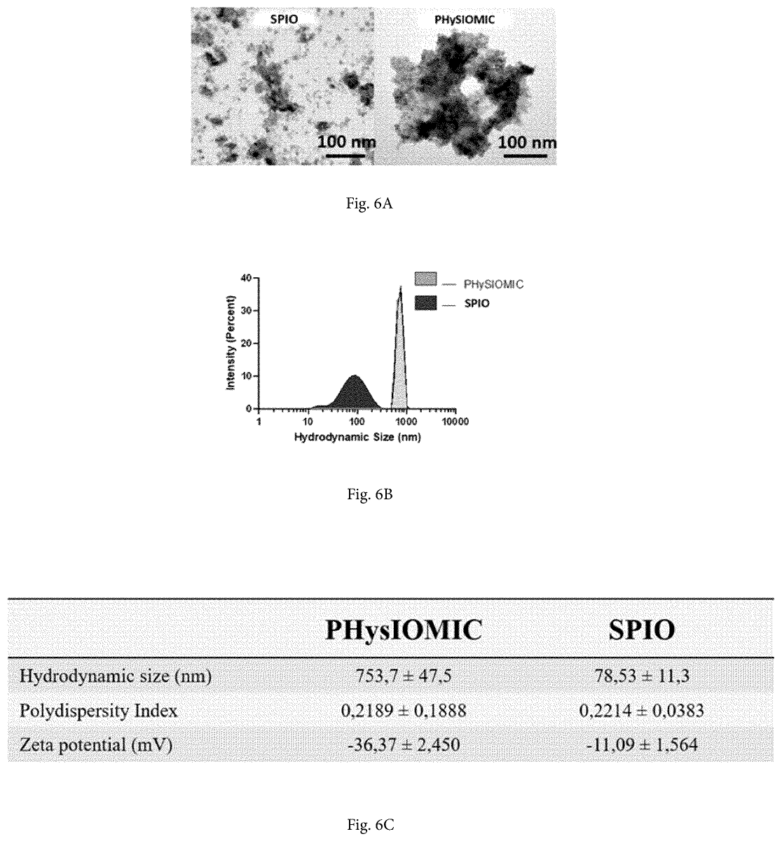

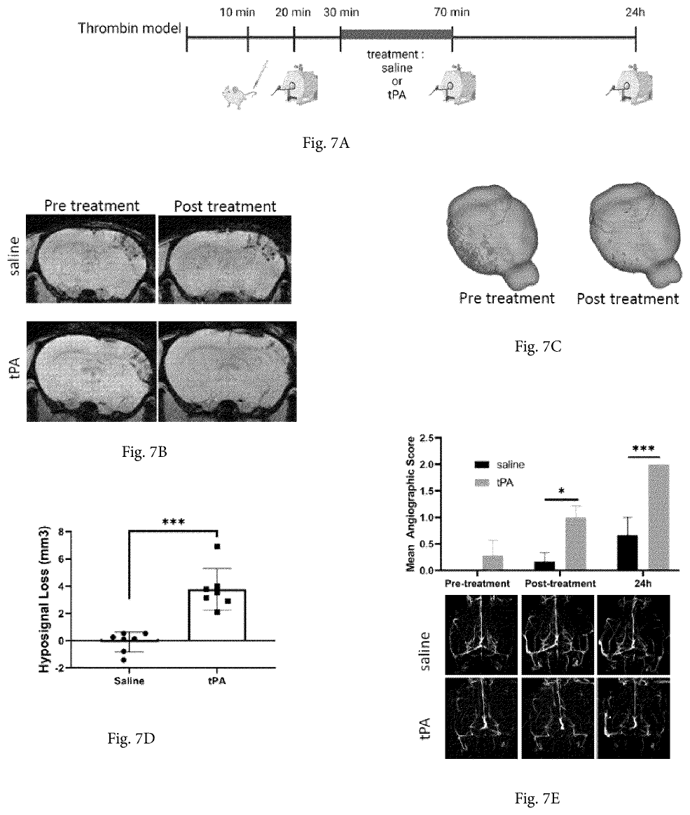

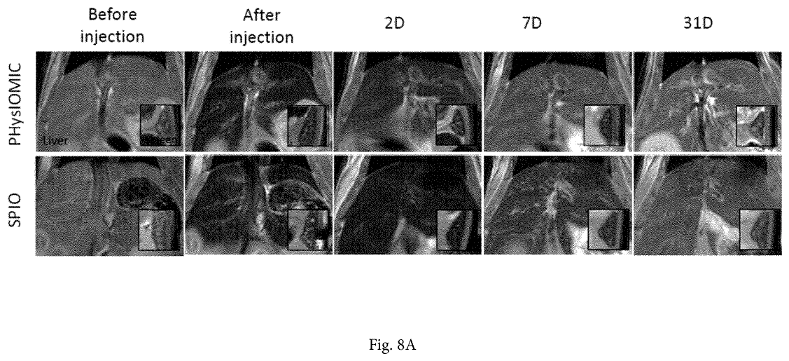

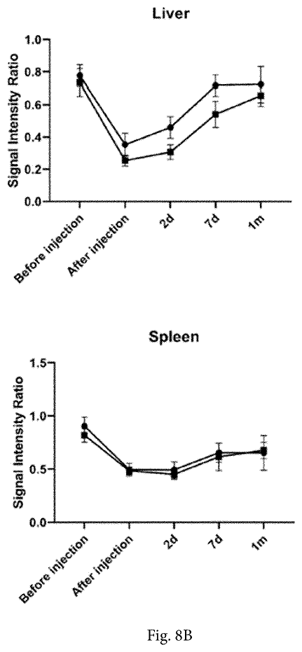

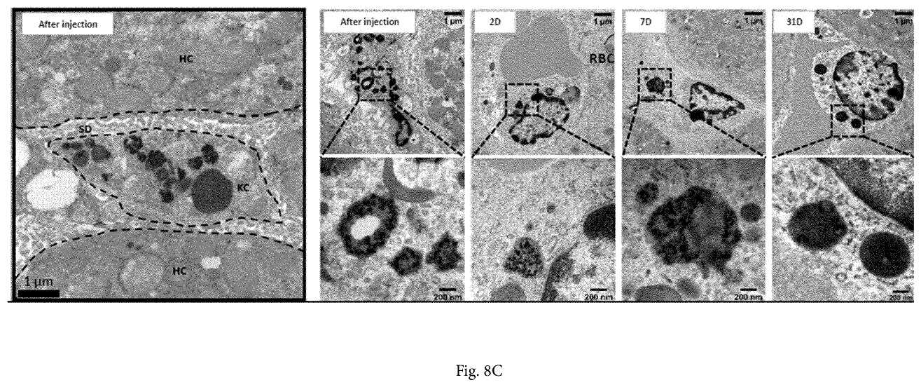

In an ischemic stroke mouse model induced via thrombin injection within the middle cerebral artery, the inventors examined the cortical microcirculation in the downstream area from the injection site by intravital bi-photon microscopy (FIG. 1). They confirmed the presence of microvascular thrombosis in this specific area as evidenced by Rhodamine 6G labelling of platelets and leukocytes. they injected FITC marked 1 μm particles intravenously and observed a specific retention at the area of the microthrombi. This experiment indicates that 1 μm diameter particles are efficient to target microemboli within the microcirculation with no need of specific targeting moiety.

### Molecular Imaging of Microthrombi in Ischemic Stroke

The inventors tested the imaging capacities of the PHysIOMIC particles in an ischemic stroke mouse model induced via thrombin injection within the middle cerebral artery; a model characterized by the formation of microvascular thrombosis in the downstream cortical microcirculation. The injection of PHysIOMICs reavealed the presence of these microthrombi on T₂* weighted MRI acquisitions (FIG. 3). The whole ischemic area corresponding to the lesion area measured at 24 h post stroke is characterized by hyposignal uptake. This signal decreases over time post injection following a kinetic profile corresponding to the spontaneous reperfusion observed in this model. Importantly, the injection of the PHysIOMICs do not worsen the stroke outcome in terms of lesion sizes. Observations on histological sections confirmed the localization of the PHysIOMICs at the edges of the microthrombi.

The PHysIOMICs were efficient to monitor thrombolysis of the microthrombi after the injection of recombinant tissue-type plasminogen activator (tPA, Actylise, 10 mg/kg) (FIG. 4). Thrombolysis with tPA significantely reduced the amount of microthrombi signal whereas saline did not interefere. The lesion size at 24 h measure by T₂ weighted MRI acquisitions was smaller for the group treated with tPA. This experiment confirms that the PHysIOMIC contrast agent can identify situation for which thrombolysis should be administered and subsequently verify the efficacy of thrombolysis therapy.

### Molecular Imaging of Brain Microthrombi Induced by Apoptosis and Ischemia-Reperfusion

The PHysIOMICs were efficient to reveal brain microthrombi in a model where thrombosis was induced by injection of Staurosporine in the striatum (FIG. 5). Signal void was observed in the area surrounding the injection site. This thrombotic situation related to apoptosis is relevant to several pathologies and present similarities with a condition called disseminated intravascular coagulation (DIC). DIC is a condition characterized by thrombosis in small blood vessels that usually develops in response to another disorder (such as cancer, sepsis, infectious disease) or event (such as organ transplant, trauma) which disrupt the coagulation system.²¹ It is for instance one of the severe complications identified in patients with pneumonia implied in infectious diseases such as the COVID-19.²² The current diagnosis method for DIC is limited to the detection of coagulation dysregulation in the blood.²³ The PHysIOMIC contrast agent can be very useful in this context, revealing on MRI scans the microthrombi present in the whole body.

The PHysIOMICs were also efficient to reveal microthrombi formed in a context of ischemia-reperfusion. The abrupt removal of the filament obstructing the middle cerebral artery for an ischemia of 60 minutes triggers the coagulation system. This situation of abrupt reperfusion is often encountered for ischemic stroke patients undergoing endovascular thrombectomy (EVT).²⁴

### EXAMPLE 2

### Material and Method

### Determination of the Hydrodynamic Diameter of the Particles

Dynamic light scattering was used to determine the average hydrodynamic diameter, the polydispersity index and the diameter distribution by volume of the SPIO used for the preparation of the PHysIOMIC particles of Example 1 and of the PHysIOMIC particles prepared according to Example 1 with a Nano ZS apparatus (Malvern Instruments, Worcestershire, UK) equipped with a 633-nm laser at a fixed scattering angle of 173°. The temperature of the cell was kept constant at 25° C., and all dilutions were performed in pure water. Measurements were performed in triplicate.

### Zeta Potential Measurement

Zeta potential analyses were realized, after 1/100 dilution in 1 mM NaCl, using a Nano ZS apparatus equipped with DTS 1070 cell. All measurements were performed in triplicate at 25° C., with a dielectric constant of 78.5, a refractive index of 1.33, a viscosity of 0.8872 centipoise, and a cell voltage of 150 V. The zeta potential was calculated from the electrophoretic mobility using the Smoluchowski equation.

### Biodistribution Study

Mice were anesthetized with isoflurane (1.5 to 2.0%) and maintained at 37° C., and PHysIOMIC or SPIO suspensions were injected intravenously (4 mg/kg). At 1 hour, 24 hours, 7 days, 1 month, and 6 months after injection, mice were perfused with saline, and a small piece of liver of approximately 1 mm³ was collected fixed with 2.5% glutaraldehyde in 0.1 M cacodylate buffer (pH 7.4).

### Whole-Body MRI

Experiments were performed using a BioSpec 7-T TEP-MRI system with a volume coil resonator (Bruker, Germany). Mice were anesthetized with isoflurane (1.5 to 2.0%) and maintained at 37° C. by the integrated heat animal holder, and the breathing rate was monitored during the imaging procedure. Whole-body scans including T2-weighted (RARE sequence, with TR/TE=3000 ms/50 ms) and T2*-weighted sequences [fast-low angle shot (FLASH) sequence, with TR/TE=50 ms/3.5 ms] were performed before, 20 min, 24 hours, 7 days, 1 month, and 6 months after the intravenous injection of SPIO and PHysIOMIC particles (4 mg/kg). Signal intensity ratios were measured by drawing the region of interest in the liver, spleen, kidney, and paravertebral muscles. Ratios were computed as the signal intensity of the organ of interest divided by the signal intensity of the paravertebral muscle (n=7).

### Transmission Electron Microscopy

For observation of SPIO or PHysIOMIC in suspension, a droplet of particles was after deposited on a hydrophilized 400-mesh grid. For observation of liver sections, the small piece harvested from the biodistribution study were dehydrated in progressive bath of ethanol (70 to 100%) and embedded in resin EMbed 812. After 20 hours of polymerization at 60° C., the coverslip were then separated from the cell's resin bloc and polymerization continued for 28 hours. Ultrathin sections were collected and contrasted with uranyl acetate and lead citrate. SPIO, PHysIOMIC and liver sections were observed with TEM JEOL 1011, and images were taken with Camera MegaView 3 and AnalySIS FIVE software.

### RESULTS AND DISCUSSION

Transmission electronic microscopy show that the PHysIOMIC particles, present a mean diameter of 753.7±47.5 nm, and are constituted by clusterized SPIO, presenting a mean diameter of 78.5+11.3 nm as shown in FIG. 6 and Table 1 below:

| | PHysIOMIC | SPIO |
|---|---|---|
| Hydrodynamic size (nm) | 753.7 ± 47.5 | 78.53 ± 11.3 |
| Polydispersity index | 0.2189 ± 0.1888 | 0.2214 ± 0.0383 |
| Zeta potential (mV) | −36.37 ± 2.450 | −11.09 ± 1.564 |

The presence of a the polydopamine matrix slightly decreases the potential zeta of the PHysIOMIC to −36.37±2.45 mV compared to the −11.09±1.56 mV, providing a favorable profile for the circulation in the blood.

The PHysIOMICs were efficient to monitor thrombolysis of the microthrombi after the injection of recombinant tissue-type plasminogen activator (tPA, Actylise, 10 mg/kg) (FIG. 7). Thrombolysis with tPA significantly reduced the amount of microthrombi signal whereas saline did not interfere. The mean angiographic score post ischemic stroke in acute setting and at 24 h post stroke confirm the beneficial effect of thrombolysis therapy. This experiment confirms that the PHysIOMIC contrast agent can identify situation for which thrombolysis should be administered and subsequently verify the efficacy of thrombolysis therapy.

The biodistribution study indicates a strong accumulation in the liver and the spleen for both SPIO and PHysIOMIC particles, as seen from the negative signal uptake after their intravenous injection (FIG. 8). A clear degradation can be observed from 7 days post injection for both types of particles. This confirms that the polydopamine matrix from the PHysIOMIC does not interfere with the biodegradability of the SPIO particles. Thus, any biocompatible SPIO that has been validated for human use could be a candidate for the preparation of PHysIOMIC particles. The transmission electronic microscopy observation of liver sections at different time post injection of PHysIOMIC particles confirms that the particles accumulate within lysosomal compartment of kupffer cells where they degrade over time.

### REFERENCES

1. Virani SS, Alonso A, Aparicio HJ, Benjamin EJ, Bittencourt MS, Callaway CW, Carson AP, Chamberlain AM, Cheng S, Delling FN, et al. Heart Disease and Stroke Statistics-2021 Update: A Report From the American Heart Association. Circulation. 2021; CIR0000000000000950.

2. Dalkara T, Arsava EM. Can Restoring Incomplete Microcirculatory Reperfusion Improve Stroke Outcome after Thrombolysis? J. Cereb. Blood Flow Metab. 2012;32:2091-2099.

3. del Zoppo GJ, Mabuchi T. Cerebral Microvessel Responses to Focal Ischemia. J. Cereb. Blood Flow Metab. 2003;23:879-894.

4. Goldberg I, Auriel E, Russell D, Korczyn AD. Microembolism, silent brain infarcts and dementia. J. Neurol. Sci. 2012;322:250-253.

5. Fransen PSS, Berkhemer OA, Lingsma HF, Beumer D, van den Berg LA, Yoo AJ, Schonewille WJ, Vos JA, Nederkoorn PJ, Wermer MJH, et al. Time to Reperfusion and Treatment Effect for Acute Ischemic Stroke: A Randomized Clinical Trial. JAMA Neurol. 2016;73:190.

6. Almekhlafi MA, Demchuk AM, Mishra S, Bal S, Menon BK, Wiebe S, Clement FM, Wong JH, Hill MD, Goyal M. Malignant Emboli on Transcranial Doppler During Carotid Stenting Predict Postprocedure Diffusion-Weighted Imaging Lesions. Stroke. 2013;44:1317-1322.

7. Belliere J, Martinez de Lizarrondo S, Choudhury RP, Quenault A, Le Béhot A, Delage C, Chauveau D, Schanstra JP, Bascands J-L, Vivien D, et al. Unmasking Silent Endothelial Activation in the Cardiovascular System Using Molecular Magnetic Resonance Imaging. Theranostics. 2015;5:1187-1202.

8. Fournier AP, Quenault A, Martinez de Lizarrondo S, Gauberti M, Defer G, Vivien D, Docagne F, Macrez R. Prediction of disease activity in models of multiple sclerosis by molecular magnetic resonance imaging of P-selectin. Proc. Natl. Acad. Sci. 2017;114:6116-6121.

9. Bonnard T, Gauberti M, Martinez de Lizarrondo S, Campos F, Vivien D. Recent Advances in Nanomedicine for Ischemic and Hemorrhagic Stroke. Stroke. 2019;50:1318-1324.

10. von zur Muhlen C, von Elverfeldt D, Moeller JA, Choudhury RP, Paul D, Hagemeyer CE, Olschewski M, Becker A, Neudorfer I, Bassler N, et al. Magnetic resonance imaging contrast agent targeted toward activated platelets allows in vivo detection of thrombosis and monitoring of thrombolysis. Circulation. 2008; 118:258-267.

11. Heidt T, Ehrismann S, Hövener J-B, Neudorfer I, Hilgendorf I, Reisert M, Hagemeyer CE, Zirlik A, Reinöhl J, Bode C, et al. Molecular Imaging of Activated Platelets Allows the Detection of Pulmonary Embolism with Magnetic Resonance Imaging. Sci. Rep. 2016;6:25044.

12. Laffon B, Fernández-Bertolez N, Costa C. Brandão F, Teixeira JP, Pásaro E, Valdiglesias V. Cellular and Molecular Toxicity of Iron Oxide Nanoparticles. Adv. Exp. Med. Biol. 2018; 1048:199-213.

13. Volatron J, Carn F, Kolosnjaj-Tabi J, Javed Y, Vuong QL, Gossuin Y, Ménager C, Luciani N, Charron G, Hémadi M, et al. Ferritin Protein Regulates the Degradation of Iron Oxide Nanoparticles. Small. 2017;13:1602030.

14. Volatron J, Carn F, Kolosnjaj-Tabi J, Javed Y, Vuong QL, Gossuin Y, Ménager C, Luciani N, Charron G, Hémadi M, et al. Ferritin Protein Regulates the Degradation of Iron Oxide Nanoparticles. Small Weinh. Bergstr. Ger. 2017;13.

15. Lee H, Dellatore SM, Miller WM, Messersmith PB. Mussel-Inspired Surface Chemistry for Multifunctional Coatings. Science. 2007;318:426-430.

16. Kim BYS, Rutka JT, Chan WCW. Nanomedicine. N. Engl. J. Med. 2010;363:2434-2443.

17. Orset C, Macrez R, Young AR, Panthou D, Angles-Cano E, Maubert E, Agin V, Vivien D. Mouse Model of In Situ Thromboembolic Stroke and Reperfusion. Stroke. 2007;38:2771-2778.

18. Karatas H, Erdener SE, Gursoy-Ozdemir Y, Gurer G, Soylemezoglu F, Dunn AK, Dalkara T. Thrombotic distal middle cerebral artery occlusion produced by topical FeCl (3) application: a novel model suitable for intravital microscopy and thrombolysis studies. J. Cereb. Blood Flow Metab. Off. J. Int. Soc. Cereb. Blood Flow Metab. 2011;31:1452-1460.

19. Longa EZ, Weinstein PR, Carlson S, Cummins R. Reversible middle cerebral artery occlusion without craniectomy in rats. Stroke. 1989;20:84-91.

20. Gauberti M, Obiang P, Guedin P, Balossier A, Gakuba C, Diependaele AS, Chazalviel L, Vivien D, Young AR, Agin V, et al. Thrombotic stroke in the anesthetized monkey (Macaca mulatta): characterization by MRI—a pilot study. Cerebrovasc. Dis. Basel Switz. 2012;33:329-339.

21. Adelborg K, Larsen JB, Hvas A-M. Disseminated intravascular coagulation: epidemiology, biomarkers, and management. Br. J. Haematol. 2021;

22. Lillicrap D. Disseminated intravascular coagulation in patients with 2019-nCOV pneumonia. J. Thromb. Haemost. JTH. 2020;18:786-787.

23. Levi M, Sivapalaratnam S. Disseminated intravascular coagulation: an update on pathogenesis and diagnosis. Expert Rev. Hematol. 2018;11:663-672.

24. Sutherland BA, Neuhaus AA, Couch Y, Balami JS, DeLuca GC, Hadley G, Harris SL, Grey AN, Buchan AM. The transient intraluminal filament middle cerebral artery occlusion model as a model of endovascular thrombectomy in stroke. J. Cereb. Blood Flow Metab. Off. J. Int. Soc. Cereb. Blood Flow Metab. 2016;36:363-369.

1-14. (canceled)

15. A particle having a hydrodynamic diameter comprised between 200 nm and 2000 nm, said particle comprising coated nanoparticles of iron oxide embedded within a matrix of polycathecolamine or polyserotonine, wherein each of said coated nanoparticles of iron oxide is coated by a polymer which is different from polycathecolamine or polyserotonine.

16. The particle according to claim 15, wherein the iron oxide is maghemite of formula Fe₂O₃, magnetite of formula Fe₃O₄ or a mixture of Fe₂O₃ and Fe₃O₄.

17. The particle according to claim 15, wherein the polycathecolamine is polydopamine (PDA), polynorepinephrine (PNE) or polyepinephrine (PEP).

18. The particle according to claim 15, wherein the polymer is a dextran or a polyethylene glycol.

19. A suspension comprising a plurality of particles according to claim 15.

20. A process of preparing a suspension of particles according to claim 19, comprising the steps of:
a) mixing by agitation a solution of catecholamine or serotonine with coated iron oxide nanoparticles, thereby causing self-polymerization of the catecholamine or the serotonine and formation of particles containing coated iron oxide nanoparticles embedded in a matrix of polymerized catecholamine or serotonine;
b) terminating said self-polymerization;
c) treating the resulting reaction mixture to obtain final particles of a desired size; and
d) recovering the suspension of particles.

21. Particles obtainable by the process according to claim 20 or a suspension comprising the particles.

22. A conjugate comprising the particle according to claim 15 and i) a molecule comprising free amine or thiol groups, or ii) a molecule comprising a radiolabelled metal.

23. The conjugate according to claim 22, wherein the molecule comprising free amine or thiol groups is a protein, a peptide, a nanobody, or a monoclonal antibody.

24. An in vivo method of imaging a patient in need thereof, comprising
administering to the patient a composition containing the particles according to claim 15, a suspension comprising the particles, or a composition comprising conjugates comprising the particles, and
performing medical imaging of the patient.

25. The in vivo method of claim 24, wherein the medical imaging is magnetic resonance imaging (MRI), magnetic particle imaging (MPI), photoacoustic imaging, or positron emission tomography (PET).

26. The in vivo method of claim 24, wherein the particles, the suspension or the conjugates are contrast agents or tracers for the in vivo method.

27. A composition containing one or more particles according to claim 15, a suspension comprising the one or more particles or a conjugate comprising the one or more particles.

28. The composition of claim 27, wherein the conjugate comprises at least one of the one or more particles and i) a molecule comprising free amine or thiol groups or ii) a molecule comprising a radiolabelled metal.

29. The particle according to claim 18, wherein the dextran is dextran, carboxydextran, or carboxymethyldextran.
